(19) United States
(12) Patent Application Publication
(10) Pub. No.: US 2017/0161434 A1
(43) Pub. Date: Jun. 8, 2017

(54) IMPLANT DATA MANAGEMENT DEVICE, SYSTEM COMPRISING THIS DEVICE AND USE OF THIS SYSTEM

(71) Applicant: Stéphane NAUDI, Perpignan (FR)

(72) Inventor: Stéphane NAUDI, Perpignan (FR)

(21) Appl. No.: 15/115,598

(22) PCT Filed: Jan. 16, 2015
(86) PCT No.: PCT/EP2015/050788

§ 371 Date: Jul. 29, 2016

(30) Foreign Application Priority Data
Feb. 4, 2014 (FR) 1450857

(51) Int. Cl. G06F 19/00; A61B 90/98; A61B 5/00; G06F 17/30

(52) U.S. Cl. CPC G06F 19/322; G06F 17/30477; G06F 17/30312; A61B 90/98; A61B 5/0031; A61B 2562/0247

(57) ABSTRACT

An implant data management device is provided, including: an electronic device to be arranged on an implant, configured to transmit identification data in response to an electrical signal, an energy transmitter arranged to be connected to an energy receiver of the electronic device arranged to receive transmitted energy and deliver the electrical signal to the electric device; a wireless receiver configured to receive the implant identification data; a processor connected to the wireless receiver; and a memory connected to the processor, the memory including a plurality of instructions. The present device is arranged to communicate with an implants database and the plurality of instructions is arranged in order to instruct the processor to: receive the implant identification data and generate an implant data search based on the implant identification data and send the generated search to the implants database.

### FIELD OF THE INVENTION

The invention relates to an implant data management device, an implant data management system utilizing this device and use of this device in this system for managing implant data.

### PRIOR ART

A system for managing patient data is known, disclosed in patent EP2263610 B1.

The system for managing patient data according to this patent comprises:
an orthopaedic implant configured to transmit implant identification data and implant sensor data in response to an electrical signal;
a primary coil configured to be inductively coupled to a secondary coil of the orthopaedic implant in order to deliver the electrical signal to the orthopaedic implant;
a wireless receiver configured to receive the implant identification data and the implant sensor data transmitted by the orthopaedic implant;
a processor connected to the wireless receiver; and
a memory device electrically connected to the processor, the memory device containing a plurality of instructions which, when executed by the processor, instruct the processor to:
   receive the implant identification data and the implant sensor data originating from the wireless receiver; and
   search for the patient data in a database on the basis of the implant identification data.

A drawback of a system for managing patient data according to this prior art is that this system utilizes sensitive data of a private and protected nature.

Another drawback of a system for managing patient data according to this prior art is that it is not arranged to access implant data directly. In fact, this prior art teaches that the system for managing data must search for patient data beforehand before determining implant data.

A purpose of the invention is to propose an implant data management device that does not utilize sensitive data of a protected and personal nature.

Another purpose of the invention is to propose an implant data management device arranged to access implant data directly.

### DISCLOSURE OF THE INVENTION

At least one of these purposes is achieved with an implant data management device comprising:
an electronic device capable of being arranged on an implant and configured to transmit implant identification data in response to an electrical signal;
energy transmission means arranged to be connected to energy receiving means of the electronic device arranged to receive transmitted energy and deliver the electrical signal to the electrical device;
a wireless receiver configured to receive the implant identification data transmitted by the electronic device;
a processor connected to the wireless receiver; and
a memory electrically connected to the processor, the memory comprising a plurality of instructions.

The implant data management device is arranged to communicate with an implants database and the plurality of instructions is arranged so that when executed by the processor, it instructs the processor to:
receive the implant identification data; and
generate an implant data search based on the implant identification data and send the generated search to the implants database.

In this description, an implant denotes an implantable medical device. The electronic device can be used in particular on orthopaedic surgery implants such as replacement hip, knee, shoulder or also ankle joints or on neurosurgical implants. The implant can be used on osteosynthesis or arthrodesis materials.

The implant data management device according to the invention therefore corresponds to the set purposes:
the system for managing implant data does not utilize protected sensitive data and data of a personal nature and,
the system for managing data is arranged to access implant data directly.

The implant data management device according to the invention requires no knowledge of patient data to identify the implant via the wireless receiver and know the history of the implant via the implant data contained in the implants database.

The energy transmission and receiving means can be means for transmitting energy in the form of a magnetic field. In particular, the energy transmission means can comprise a primary coil and the energy receiving means of the electronic device can comprise a first secondary coil.

The energy transmission and receiving means can also be means for transmitting energy in the form of an electric field. In particular, the energy transmission and receiving means can comprise capacitive electrodes.

The energy transmission and receiving means can also be means for transmitting energy in the form of electromagnetic waves. In particular, the energy transmission means can comprise one antenna and the energy receiving means can comprise another antenna.

In a preferred embodiment, the electronic device is implemented in the form of an RFID (Radio Frequency Identification) tag. This technology makes it possible to identify an object, to track it and recognize its characteristics remotely thanks to a tag emitting radio waves, attached to or incorporated in an object. RFID technology makes it possible to read tags even without a direct line of sight and can pass through thin layers of materials, such as those constituted by a human tissue.

More preferentially, the RFID tag is a passive RFID tag. Passive RFID tags use the energy propagated over a short distance by the radio signal of the wireless receiver. Preferably, RFID tags have no permanent internal of source of energy, such as a battery. These tags are generally smaller than active RFID tags and have a virtually unlimited lifetime.

Preferably, the connection between the processor and the wireless receiver is achieved via a wireless link. The effect of this wireless link is to make it possible to incorporate the wireless receiver in a casing remote from a device comprising the processor. This wireless link can comply with a telecommunication standard, such as the standard IEEE 802.11 or the standard IEEE 802.15. In a variant, the processor and the wireless receiver can be incorporated in the same casing, for example a laptop or also a tablet.

Advantageously, the device according to the invention can also comprise at least one sensor arranged inside or in contact with the implant, the at least one sensor being configured to transmit sensor data in response to an electrical signal, the wireless receiver also being configured to receive the sensor data from the at least one sensor. The arrangement of the sensor has the effect of improving the quality of the data captured by the sensor.

Preferably and when it comprises at least one such sensor, the electronic device is arranged to deliver an electrical signal to the at least one sensor when an electrical signal is delivered to the electronic device. In this case, the electronic device is capable of transferring transmissions of sensor data.

Advantageously and when it comprises at least one such sensor, this sensor can comprise a second energy receiving means arranged to receive the energy transmitted by the energy transmission means and deliver an electrical signal to the at least one sensor.

The second energy receiving means can be a transmitted energy receiving means in the form of a magnetic field. In particular, the second energy receiving means can be a second secondary coil.

The second energy receiving means can also be a means for receiving energy transmitted in the form of an electric field. In particular, the second energy receiving means can comprise capacitive electrodes.

The second energy receiving means can also be a means for receiving energy transmitted in the form of electromagnetic waves. In particular, the second energy receiving means can comprise another antenna.

The presence of the second energy receiving means allows this sensor to have an energy supply additional to that which can be delivered by the electronic device. A supply to this sensor independent of that of the electronic device makes it possible to dispense with a supply connection between the first secondary coil and this sensor. When the electronic device is arranged to deliver an electrical signal to this sensor, a redundant supply to this sensor is thus obtained and this makes it possible to ensure the continuity of the electrical supply to this sensor.

Preferably, the electronic device comprises the implant identification data and the energy receiving means.

Advantageously and when the device according to the invention comprises at least one such sensor, this sensor and the electronic device can be arranged to be physically incorporated on the implant. Of course, when the device according to the invention comprises no such sensor, the electronic device alone can be arranged to be physically incorporated on the implant.

Advantageously and when the device according to the invention comprises at least one such sensor, this sensor can be a pressure sensor. Of course, another type of sensor can be envisaged, such as a temperature or vibration sensor, or also a biological or biomechanical sensor for assessing quantities of bacteria or viruses.

When the device according to the invention comprises a pressure sensor, the pressure sensor can be produced in the form of a microelectromechanical system. A microelectromechanical system is a microsystem comprising one or more mechanical elements, using electricity as an energy source, for carrying out a sensor and/or actuator function, with at least one structure having micrometric dimensions; the function of the system being in part ensured by the form of this structure. The term microelectromechanical system is referred to by the acronym MEMS.

Advantageously, the plurality of instructions can also be arranged, when executed by the processor, to instruct the processor to generate a completion of the implant data on the basis of the implant identification data and send the completion generated to the implant database. The completion generated can comprise for example the date of the reading carried out by the wireless receiver. When the device according to the invention comprises at least one sensor as described above, the completion generated can comprise for example the date of the reading carried out by the wireless receiver as well as the sensor data received by the wireless receiver in response to an electrical signal.

Preferentially, the device according to the invention comprises a volatile memory and the implant identification data are stored in the volatile memory. A volatile memory is a computer memory which needs a continuous power supply to retain the information recorded therein. When the power supply is interrupted, the information contained in the volatile memory is immediately lost. The volatile memory is different from the previously described memory connected to the processor.

More preferentially, the implant data are never stored in a non-volatile memory. The term read-only memory is also used for a non-volatile memory, as opposed to a random-access memory which is a volatile memory.

According to another aspect of the invention, an implant data management system is proposed comprising:
at least one implant data management device according to the invention,
an implants database capable of communicating with the at least one implant data management device.

Preferably and when the plurality of instructions for the at least one data management device according to the invention comprises a completion of the implant data, the completion of implant data of the at least one implant data management device can be sent to the implants database via a computer network.

Advantageously, the system can also comprise at least one terminal comprising a display, the at least one terminal being arranged to:
send an implant data search to the implants database, the search being based on the implant identification data,
display results generated by the implants database after the generated search is received.

Preferentially, the implant data search can be generated by the at least one implant data management device.

According to another aspect of the invention, a use of at least one management device according to the invention within a system according to the invention is proposed.

Preferably, the wireless receiver of the at least one management device utilizes a plurality of receptions of sensor data, these sensor data varying as a function of a mechanical load applied to the implant of the data management device. It is thus possible to generate at least one curve from this plurality of receptions of sensor data. This curve is a load curve when the plurality of receptions of sensor data changes in accordance with an increase in the mechanical load applied to the implant. This curve is an unload curve when the plurality of receptions of sensor data changes in accordance with a reduction in the mechanical load applied to the implant.

Preferably, when the use in fact comprises utilization of a plurality of receptions of sensor data as described above and when the at least one management device is arranged to generate a completion of the implant data, the completion of the implant data can comprise data from the plurality of receptions of sensor data. Thus, the implants database comprises, for each implant, a history for this implant of implant data constituted for example by a date of the reading by the wireless receiver of the device according to the invention, of implant data comprising implant identification data and pluralities of sensor data receptions.

Advantageously, the use according to the invention can be implemented in order to carry out a longitudinal analysis of the condition of the implant. The longitudinal analysis of the condition of the implant is carried out by interrogating the database which contains a history for each implant. Changes in the implant data of this history allow analysis of the condition of the implant. This analysis can for example result in a level of probability of loosening of the implant under certain conditions.

### DESCRIPTION OF THE FIGURES AND EMBODIMENTS

Other advantages and characteristics of the invention will become apparent on examination of the detailed description of an embodiment which is in no way limitative, and the following attached diagrams:

FIG. 1 shows a preferred embodiment of a system according to the invention comprising a plurality of devices according to the invention;

FIG. 2 shows in more detail one of the devices according to the invention present in the system shown in FIG. 1.

As this embodiment is in no way limitative, variants of the invention can be considered comprising only a selection of the characteristics described hereinafter, in isolation from the other characteristics described (even if this selection is isolated within a phrase containing other characteristics), if this selection of characteristics is sufficient to confer a technical advantage or to differentiate the invention with respect to the state of the art. This selection comprises at least one, preferably functional, characteristic without structural details, or with only a part of the structural details if this part alone is sufficient to confer a technical advantage or to differentiate the invention with respect to the state of the prior art.

In the figures an element appearing in several figures keeps the same reference.

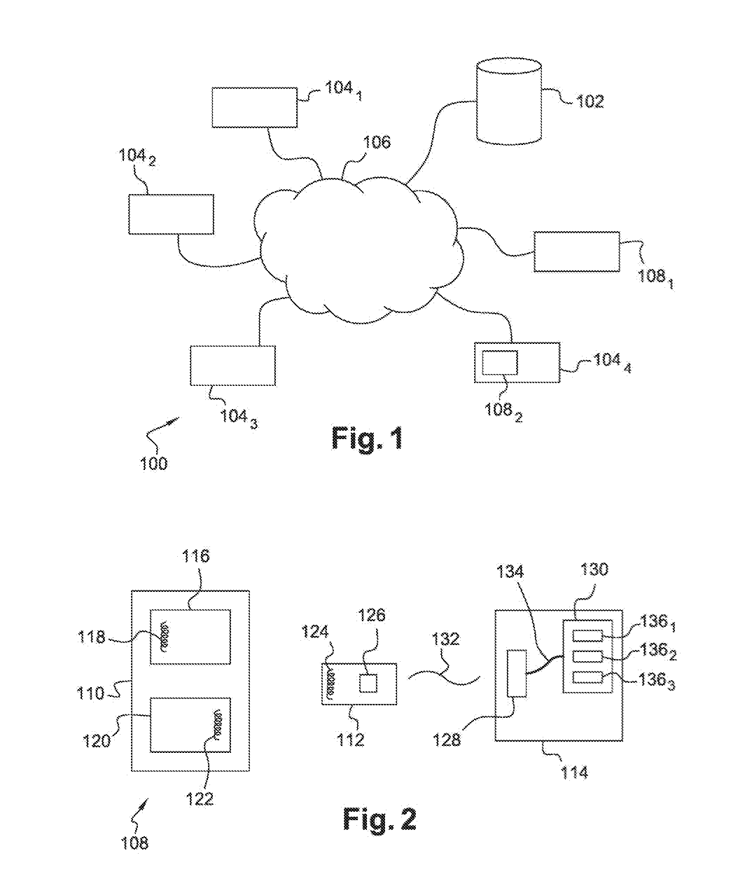

A preferred embodiment of an implant data management system 100 according to the invention will now be described with reference to FIG. 1.

The implant data management system 100 comprises:
an implants database 102,
four implant data management devices according to the invention 104₁, 104₂, 104₃, 104₄,
a diagrammatic representation of a computer network 106.

The computer network 106 links the implants database 102 to the four implant data management devices 104₁, 104₂, 104₃, 104₄.

The implant data management system 100 also comprises two terminals 108₁ and 108₂, each of the terminals 108₁ and 108₂ comprising a display. The computer network 106 links the implants database 102 and the four implant data management devices 104₁, 104₂, 104₃, 104₄ to the terminal 108₁. The terminal 108₂ is included in the implant data management device 104₄. The terminals 108₁ and 108₂ are described below.

The implants database 102 is capable of communicating with the four implant data management devices 104₁, 104₂, 104₃, 104₄. In this embodiment, the implants database 102 communicates with the four implant data management devices 104₁, 104₂, 104₃, 104₄ via the computer network 106. The computer network 106 is of the internet type. Of course each implant data management device 104₁, 104₂, 104₃, 104₄ can be linked to a network hub, the network hub being linked to the computer network 106. Of course, any other network topology and/or physical communication method can be envisaged, such as wired or wireless communication.

It will be noted that the four implant data management devices 104₁, 104₂, 104₃, 104₄ are thus physically separate from the implants database 102, which centralizes the information collected by the four implant data management devices 104₁, 104₂, 104₃, 104₄.

An implant data management device 108 according to the invention will now be described in more detail with reference to FIG. 2. It should be recalled here that the implant data management devices 104₁, 104₂, 104₃, 104₄ are implant data management devices according to the invention.

The implant data management device 108 comprises:
an implant module 110,
a reader casing 112,
a control computer 114.

The implant module 110 is capable of being arranged on an implant.

The implant module 110 comprises:
an electronic device 116,
a first secondary coil 118 of the electronic device 116,
a sensor 120 arranged within or in contact with the implant.

The electronic device 116 is configured to transmit implant identification data in response to an electrical signal.

The sensor 120 is a pressure sensor produced in the form of a microelectromechanical system.

The sensor 120 comprises a second secondary coil 122 and is configured to transmit sensor data in response to an electrical signal. The arrangement of the sensor has the effect of improving the quality of the data captured by the sensor.

The electronic device 116 is utilized in the form of a passive RFID tag. Thus, the electronic device 116 comprises the implant identification data and the first secondary coil 118.

The presence of the second secondary coil 122 allows the sensor 120 to have a power supply additional to which that which could be delivered to it by the electronic device 116. In this embodiment, the supply to this sensor 120 is independent of that from the electronic device 116. This independent supply makes it possible to dispense with a supply connection between the first secondary coil 118 and the sensor 120.

The sensor 120 and the electronic device 116 are arranged to be physically incorporated on the implant.

The reader casing 112 comprises:
a primary coil 124,
a wireless receiver 126.

The primary coil 124 is configured to be coupled by induction to the first secondary coil 118 of the electronic device 116 in order to deliver the electrical signal to the electronic device 116.

The primary coil 124 is also configured to be coupled by induction to the second secondary coil 122 in order to deliver an electrical signal to the sensor 120.

The wireless receiver 126 is configured to receive the implant identification data transmitted by the electronic device 116. The wireless receiver 126 is also configured to receive the sensor data from the sensor 120.

The control computer 114 comprises:
a processor 128,
a memory 130.

The processor 128 is connected to the wireless receiver 126 via a wireless link 132. The effect of this wireless link 132 is to allow the wireless receiver 126 to be incorporated in the reader casing 112 remote from the control computer 114 comprising the processor 128. This wireless link 132 complies with telecommunication standard IEEE 802.15, better known by the name of Bluetooth.

The memory 130 is electrically connected to the processor 128 via an electrical link 134. The memory comprises a plurality of instructions 136₁, 136₂, 136₃.

The implant data management device 108 is arranged to communicate with the implants database 102 of FIG. 1.

The plurality of instructions 136₁, 136₂, 136₃, is arranged so that, when executed by the processor 128, it instructs the processor 128 to:
receive the implant identification data; and
generate an implant data search based on the implant identification data and send the generated search to the implants database 102 of FIG. 1.

The plurality of instructions 136₁, 136₂, 136₃ is also arranged so that, when executed by the processor 128, it instructs the processor 128 to generate a completion of the implant data on the basis of the implant identification data and send the completion generated to the implant database. The completion generated comprises the date of the reading carried out by the wireless receiver 126 as well as the sensor data received by the wireless receiver 126 in response to an electrical signal.

The completion of implant data from the implant data management device 108 is sent to the implants database 102 of FIG. 1 via a computer network 106 of FIG. 1.

In the device 108 according to the invention, the implant identification data are never stored in a non-volatile memory. Thus, no information relating to a patient is collected or retained either within the implant module 110, or within the reader casing 112, or by the control computer 114, or by the database 102. Moreover the reader casing 112 and the control computer 114 have no memory of the collected implant data.

The terminals 108₁ and 108₂ shown in FIG. 1 are capable of communicating with the implants database 102 and are arranged to:
send an implant data search to the implants database 102, the search being based on the implant identification data,
display results generated by the implants database 102 after the generated search is received.

Of course, the functions carried out by the processor 128 can be controlled by software stored in the memory 130. This software can also be produced in the form of a website. The implant data management system 100 would in this case have a web server for providing the software in the form of a website.

According to a first variant of a second embodiment of a device according to the invention, only described where it differs from the preferred embodiment of a device according to the invention, the electronic device 116 is arranged to deliver an electrical signal to the sensor 120 when an electrical signal is delivered to the electronic device 116. In this case, the electronic device 116 is capable of transferring transmissions of sensor data. There is then no longer any need for a second secondary coil 122.

According to a second variant of this second embodiment, only described where it differs from the first variant of the second embodiment, this second variant has a secondary coil 122, configured as in the first embodiment and capable of supplying the sensor 120. A redundant supply to the sensor 120 is thus obtained and this makes it possible to ensure the continuity of the electrical supply to this sensor 120.

According to a third embodiment, only described where it differs from the first and optionally combinable with the two presented variants of the second embodiment, the elements of the casing 112 and of the control computer 114 can be incorporated in the same casing, for example a laptop or also a digital tablet.

A preferred use of a management device according to the invention within a system according to the invention is now described.

In this use, a wireless receiver 126 of an implant management device utilizes a plurality of receptions of sensor data. These sensor data vary as a function of a mechanical load applied to the implant.

It is thus possible to generate at least one curve from this plurality of receptions of sensor data. This curve is called a load curve when the plurality of receptions of sensor data changes in accordance with an increase in the mechanical load applied to the implant. This curve is called an unload curve when the plurality of receptions of sensor data changes in accordance with a reduction in the mechanical load applied to the implant. It is thus possible to generate a curve representative of load and unload cycles.

The completion of the implant data comprises data from the plurality of receptions of sensor data. It also comprises a representative measurement of load and unload cycles. Thus, the implants database 102 comprises, for each implant, a history for this implant of implant data constituted by a date of the reading of the implant identification data by the wireless receiver of the implant data management device 104₁, 104₂, 104₃, 104₄, and the plurality of receptions of sensor data.

This use is implemented in order to carry out a longitudinal analysis of the condition of the implant. The longitudinal analysis of the condition of the implant is carried out by interrogating the database 102 which contains a history for each implant. In particular, a longitudinal analysis of representative measurements of load and unload cycles of this history allows analysis of the condition of the implant. This analysis can for example conclude a level of probability of loosening of the implant under certain conditions. The teaching of the longitudinal analysis of representative measurements of load and unload cycles is more informative than that of the static analysis of load and/or unload measurements under non-standardized conditions. This teaching has the advantage of allowing a better prediction of a loosening of the implant.

Of course, the invention is not limited to the examples which have just been described and numerous adjustments can be made to these examples without exceeding the scope of the invention.

1-19. (canceled)

20. An implant data management device comprising:
an electronic device arrangable on an implant and configured to transmit implant identification data in response to an electric signal;
energy transmission means arranged to be connected to energy receiving means of the electronic device arranged to receive transmitted energy and deliver the electric signal to the electric device;
a wireless receiver configured to receive the implant identification data transmitted by the electronic device;
a processor connected to the wireless receiver; and
a memory electrically connected to the processor, the memory comprising a plurality of instructions;
at least one sensor arranged within or in contact with the implant, the at least one sensor being configured to transmit sensor data in response to an electric signal, the wireless receiver also being configured to receive the sensor data from said at least one sensor,
in which the implant data management device is arranged to communicate with an implants database and in which the plurality of instructions is arranged so that, when executed by the processor, it instructs the processor to:
receive the implant identification data;
generate an implant data search based on the implant identification data and send said generated search to said implants database; and
generate a completion of the implant data comprising said sensor data on the basis of the implant identification data and send said generated completion to the implants database.

21. The device according to claim 20, characterized in that the energy transmission means comprise a primary coil and in that the energy receiving means of the electronic device comprise a first secondary coil.

22. The device according to claim 20, in which the electronic device is implemented in the form of an RFID label.

23. The device according to claim 20, in which the connection between the processor and the wireless receiver is achieved via a wireless connection.

24. The device according to claim 20, in which the electronic device is arranged to deliver an electric signal to the at least one sensor when an electric signal is delivered to the electronic device.

25. The device according to claim 20, in which the sensor comprises a second energy receiving means arranged to receive energy transmitted by the energy transmission means and deliver an electric signal to the at least one sensor.

26. The device according to claim 20, in which the at least one sensor and the electronic device are arranged to be physically incorporated on the implant.

27. The device according to claim 20, in which at least one of the sensors is a pressure sensor.

28. The device according to claim 27, in which the at least one pressure sensor is produced in the form of a microelectromechanical system.

29. An implant data management system comprising:
at least one implant data management device according to claim 20; and
an implants database capable of communicating with said at least one implant data management device.

30. The system according to claim 29, in which the completion of the implant data of the at least one implant data management device is sent to the implants database via a computer network.

31. The system according to claim 29, the system also comprising at least one terminal comprising a display, the at least one terminal being arranged to:
send an implant data search to the implants database, the search being based on the implant identification data; and
display results generated by said implants database after said generated search is received.

32. The system according to claim 31, in which the implant data search is generated by the at least one implant data management device.

33. A use of a system according to claim 29.

34. The use according to claim 33, in which the wireless receiver of the at least one management device utilizes a plurality of receipts of sensor data, these sensor data varying as a function of a mechanical load applied to the implant of the device for managing data.

35. The use according to claim 34 when dependent on claim 29, in which the completion of the implant data comprises data from the plurality of receipts of sensor data.

36. The use according to claim 33, the use being implemented to carry out a longitudinal analysis of a condition of the implant.
